(19) United States
(12) Patent Application Publication
(10) Pub. No.: US 2010/0047762 A1
(43) Pub. Date: Feb. 25, 2010

(54) METHOD TO MONITOR DRUG EFFICACY IN DIABETIC PATIENTS USING AN ASSAY FOR 1,5-ANHYDRO-D-GLUCITOL

(75) Inventors: Eric A. Button, Kannapolis, NC (US); Hirotaka Ishibashi, New York, NY (US); R. Scott Foster, Kannapolis, NC (US); Toshio Tanabe, Tokyo (JP)

Correspondence Address: DARBY & DARBY P.C., P.O. BOX 770, Church Street Station, New York, NY, 10008-0770, US

(73) Assignees: NIPPON KAYAKU KABUSHIKI KAISHA, Tokyo (JP); TOYOTA TSUSHO AMERICA, INC., New York, NY (US)

(21) Appl. No.: 12/531,426

(22) PCT Filed: Mar. 20, 2008
(86) PCT No.: PCT/US08/57694

§ 371 Date: Sep. 21, 2009

Related U.S. Application Data
(60) Provisional application No. 60/895,976, filed on Mar. 20, 2007.
(60) Provisional application No. 60/896,233, filed on Mar. 21, 2007.

(51) Int. Cl. C12Q 1/00

(52) U.S. Cl. 435/4

(57) ABSTRACT

HbA1c measurement is a critical component of diabetes management; however, a key limitation of HbA1c as a measure of glycemia is the lack of timeliness—it does not detect underlying blood glucose excursion levels in moderately controlled diabetic patients (HbA1c<8) as it is a measurement of mean glucose levels over the longer-term. HbA1c also averages both hypo- and hyperglycemia over two to three months; therefore, it does not adequately reflect improvements in post-prandial hyperglycemia. 1,5-AG is also a marker of glycemic control over a shorter one to two week timeframe, but with a different mechanism than HbA1c. Given the unique biological and physiological characteristics of 1,5-AG, it is sensitive to acute and transient episodes of hyper-glycemia and is, therefore, a better indicator of glucose excursions. Peptidyl diabetic drugs such as pramlintide and exenatide have unique mechanisms of action and the glycemic effects of these drugs are not adequately shown by HbA1c. 1,5-AG, an effective measure of glucose excursions, reveals underlying treatment effects of these drugs and can help regulate their dosage.

This application claims priority to U.S. Provisional Application Nos. 60/895,976, filed Mar. 20, 2007 and 60/896,233, filed Mar. 21, 2007, the entire contents of which are incorporated hereby by reference.

### BACKGROUND OF THE INVENTION

The importance of tight glycemic control to prevent diabetic complications has been well accepted. Recent studies indicate that postprandial glucose is an independent risk factor for the development of microvascular and macrovascular complications. Many well controlled patients with diabetes have significant postprandial hyperglycemia. For that reason, new drugs targeting strict control of total hyperglycemia and postprandial hyperglycemia are under development. Several drugs with new mechanisms of action, including pramlintide and exenatide, have been developed and launched.

There are several diabetic control markers, including hemoglobin A1c (HbA1c), 1,5-anhydro-D-glucitol (1,5-AG), fructosamine (FR) and glucosylated albumin (GA). HbA1c is the most popular marker in the evaluation of the effect of diabetic drugs. HbA1c is one hemoglobin fraction known as glucosylated hemoglobin. It is formed in a non-enzymatic pathway by hemoglobin's normal exposure to high plasma levels of glucose and accumulated in blood cells. It is well recognized that the level of HbA1c is proportional to mean glucose concentration for two to three months. HbA1c has several weaknesses in the evaluation of treatment effect of diabetic drugs. HbA1c is not suitable for evaluation of treatment effects in the short-term and cannot detect excursions of blood glucose levels. Furthermore, low HbA1c values may occur with sickle cell anemia, chronic renal failure and in pregnancy.

Serum 1,5-anhydro-D-glucitol is inversely affected by serum glucose above the renal threshold (180 mg/dL); therefore, lowering serum 1,5-AG levels (less than 10 μg/ml) indicate increasingly higher serum glucose concentrations. Measurement of serum 1,5-AG reflects all post-prandial (post-meal) glucose above the renal threshold over a one to two week timeframe.

### BRIEF DESCRIPTION OF FIGURES AND TABLES

FIG. 1 —Study Design. This study involves a group of patients (n=37, age 40+/−12 years, %, weight 85.9+/−20.8 kg). With a baseline HbA1c of 7.5+/−0.3 who have been treated with pramlintide (30/60 μg) or placebo with major meals.

FIG. 2 A, B, and C—Changes in HbA1c, insulin use and body weight from baseline to Week 29. FIG. 2A shows the change from baseline in HbA1c found in both a placebo group (N=19) and a pramlintide treated group of diabetes patients. FIG. 2B demonstrates the changes in insulin usage for both rapid-acting and regular insulin usage in both the placebo and pramlintide treated patients. FIG. 2C presents the changes in body weight in the placebo and pramlintide treated patients.

FIG. 3—Changes in PPG excursions from baseline Week 29. The changes in postprandial glucose (PPG) excursions is demonstrated for a placebo treated group (n=19) and a pramlintide treated group of type 1 diabetes patients (N=18).

FIGS. 4 A and B—Absolute and relative changes in 1,5-AG from baseline to Week 29. The changes in 1,5-anhydro-D-glucitol (1,5-AG) are significantly different between the placebo and the pramlintide-treated type 1 diabetes patients. FIGS. 4A and 4B show the absolute and percentage changes, respectively, for 1,5-AG after 29 weeks of treatment.

Table 1 lists non-limiting examples of amylin analogs.

Table 2 lists non-limiting examples of GLP-1 analogs.

Table 3 lists non-limiting examples of alpha-glucosidase inhibitors.

Table 4 lists non-limiting examples of dipeptidyl peptidase IV inhibitors.

Table 5 lists non-limiting examples of insulin secretagogues.

Table 6 compares the baseline characteristics of patients treated with either a placebo or pramlintide.

Table 7 summarizes the parameter changes in patients with HbA1c less than or equal 8.0%.

Table 8 presents the demographics and baseline characteristics of the study group.

Table 9 presents the study to assess the utility of 1,5-anhydro-D-glucitol, HbA1c and fructosamine to demonstrate the efficacy of exenatide.

### SUMMARY OF THE INVENTION

The present invention provides a method for determining the effect of one or more antihyperglycemia diabetes treatment drugs on a person in need of such treatment. This method includes: (a) measuring the 1,5-anhydro-D-glucitol (1,5-AG) level of the patient to obtain a first 1.5-AG level; (b) administering one or more antihyperglycemia drugs to said patient; and (c) measuring the 1,5-AG level of said patient after step (b) to obtain a second 1,5-AG level; wherein the effect of the one or more drugs is not reflected by mean HbA1c values; and wherein an increase of the second 1,5-AG level over the first 1,5-AG level indicates a positive effect of the one or more drugs. Similarly, a decrease of the second 1,5-AG level over the first 1,5-AG level indicates a negative effect of the one or more drugs. Preferably, the one or more drugs are peptide drugs, and more preferably, they are selected from the group consisting of amylin, an amylin receptor agonist, a glucagon-like peptide 1 or active fragment thereof, a glucogon-like peptide 1 receptor agonist, and, preferably, the one or more drugs are non-peptide drugs, and more preferably, they are selected from the group consisting of alpha-glucosidase inhibitor, dipeptidyl peptidase IV inhibitor, or insulin secretagogue or any combination of any of the foregoing. The patient can also be undergoing insulin therapy. These steps can be repeated more than once in sequence to determined increased or decreased effects.

The present invention also provides a method of evaluating treatment by one or more antihyperglycemia drugs selected from the group consisting of amylin, an amylin receptor agonist, glucagon-like peptide 1 or active fragment thereof, a glucogon-like peptide 1 receptor agonist or any combination of any of the foregoing, to a patient suffering from diabetes mellitus. This method includes (a) measuring the 1,5-AG level of the patient to obtain a first 1,5-AG level; (b) administering the one or more drugs to the patient; and (c) measuring the 1,5-AG level of said patient after step (b) to obtain a second 1,5-AG level; wherein an increase of the second 1,5-AG level over the first 1,5-AG level indicates a positive effect of said one or more drugs. Similarly, a decrease of the second 1,5-AG level over the first 1,5-AG indicates a negative effect of the one or more drugs. The patient can also be undergoing insulin therapy. These steps can be repeated more than once in sequence to determined increased or decreased effects.

The present invention further provides a method of determining the desired dosage of one or more antihyperglycemia drugs selected from the group consisting of amylin, an amylin receptor agonist, glucagon-like peptide 1 or active fragment thereof, a glucogon-like peptide 1 receptor agonist or any combination of any of the foregoing to be administered to a patient suffering from diabetes mellitus. This method includes (a) administering a first predetermined dosage of the one or more drugs to the patient; (b) measuring the 1,5-AG level of said patient after step (a) to obtain a first 1,5-AG level: (c) administering a second predetermined dosage of the same one or more drugs to said patient; and (d) measuring the 1,5-AG level of said patient after step (c) to obtain a first 1,5-AG level; wherein an increase of the second 1,5-AG level over the first 1,5-AG level indicates that the second predetermined dosage preferred over the first predetermined dosage for the patient. Similarly, a decrease of the second 1,5-AG level over the first 1,5-AG level indicates a negative effect of the one or more drugs. The patient can also be undergoing insulin therapy. These steps can be repeated more than once in sequence to determined increased or decreased effects. These steps can be repeated more than once in sequence to determined increased or decreased effects and to titrate to optimal dosages for the patient.

### DETAILED DESCRIPTION OF THE INVENTION

1,5-anhydro-D-glucitol (“1,5-AG”) is a monosaccharide derived from the ingestion of foods. It is a naturally occurring dietary polyol, has a similar chemical structure to glucose, and is present in human cerebrospinal fluid and plasma. Its quantity in plasma is stable in healthy subjects and is reduced in those with certain diseases, particularly with diabetes. Normally, intake and excretion of 1,5-AG are balanced. Since, 1,5-AG serum levels remain constant in normal individuals. High levels of urinary glucose block 1,5-AG readsorption in the proximal renal tubules due to the similarity between glucose and 1,5-AG. This results in increased excretion of 1,5-AG and decreased 1,5-AG serum levels. This means that 1,5-AG serum levels fall when glucose levels are elevated and when glucosuria occurs and that 1,5-AG levels are inversely proportional to the degree of hyperglycemia.

Clinically, 1,5-AG in plasma or serum can be measured conveniently by a commercial kit based on colorimetric enzymatic method using an enzyme that oxidizes 1,5-AG. Plasma levels of 1,5-AG fall as urinary glucose appears, generally at around 180 mg/dL, which is the recognized American Diabetes Association average renal threshold for glucose and the upper limit of normal postprandial glucose. Clinically, 1,5-AG can be used as a marker of postprandial hyperglycemia in patients with HbA1c levels below approximately 8%. Lower concentrations indicate glucose excursions above approximately 200 mg/dL. Thus, the 1,5-AG test respond sensitively and rapidly to serum glucose levels, reflecting even transiently ascending serum glucose above the renal threshold for glucosuria within a few days. Since 1,5-AG recovers to normal plasma levels at a constant rate, depending on the severity of the post-meal episode, hyperglycemia is measurable over the previous one to two weeks. Therefore, in contrast with HbA1c, 1,5-AG is suitable for short-term evaluation and can exclusively detect hyperglycemic excursions over a one to two week timeframe. (Diabetes Care 2004; 27:1859-1865, Diabetes Care 2006; 29: 1214-1219, WO 2006/116083 A2).

One suitable assay for 1,5-AG is the assay sold under the trademark Glycomark™ by The Biomarker Group—Kannapolis, N.C. and available through Quest, LabCorp, Esoterix, Specialty Laboratories, or Doctors Laboratory.

The term “peptide drug” means a peptide with an agonist activity or activities for hormonal receptors that are targets for the development of diabetic drugs, but it does not include insulin itself or insulin analogs. For example, peptide drugs include: (1) incretin hormones, including glucose-dependent insulinotropic polypeptide (GIP) and glucagon-like peptide-1 (GLP-1), and the analogs or portion of the peptides that can cause an increase in the amount of insulin release when glucose levels are elevated, (2) insulin-supportive hormones for postprandial glucose control, like amylin, and the analogs or portion of the peptides (3) hormones that can release resistance for insulin action, like adiponectin, and the analogs or portion of the peptides (4) appetite-suppressive hormone, like leptin, and the analogs or portion of the peptides and (5) other peptide hormones with useful features for glycemic control of diabetic patients.

Amylin is a naturally occurring neuroendocrine hormone synthesized by pancreatic beta cells that contributes to glucose control during the postprandial period.

The term “amylin receptor agonist” includes every therapeutic drug that shows agonistic activity for the amylin receptors. Preferably, such agonists include amylin itself, amylin analogs, and any synthetic peptides that show agonistic activity for the amylin receptors. Table 1 lists non-limiting examples of amylin analogs. Pramlintide (brand name, SYMLIN®) is one of amylin receptor agonist used as antihyperglycemia drug for type I diabetes patients with postprandial glucose excursions. It is typically used with insulin treatment. Pramlintide is a synthetic analog of human amylin and provided as an acetate salt of the synthetic 37-amino acid polypeptide, which differs in amino acid sequence from human amylin by replacement with proline at positions 25 (alanine), 28 (serine), and 29 (serine). Pramlintide has the following mechanisms of action by acting as an amylinomimetic agent: (1) Modulation of gastric emptying: Gastric-emptying rate is an important determinant of the postprandial rise in plasma glucose. Pramlintide slows the rate at which food is released from the stomach to the small intestine following a meal, and thus, it reduces the initial postprandial increase in plasma glucose. This effect lasts for approximately 3 hours following Pramlintide administration. Pramlintide does not alter the net absorption of ingested carbohydrate or other nutrients; (2) Prevention of the postprandial rise in plasma glucagon: In patients with diabetes, glucagon concentrations are abnormally elevated during the postprandial period, contributing to hyperglycemia. Pramlintide has been shown to decrease postprandial glucagon concentrations in insulin-using patients with diabetes; (3) Satiety leading to decreased caloric intake and potential weight loss: Pramlintide administered prior to a meal has been shown to reduce total caloric intake. This effect appears to be independent of the nausea that can accompany Pramlintide treatment. In a clinical study on pramlintide, dose escalation of pramlintide with reduced mealtime insulin was effective during therapy initiation in patients with type 1 diabetes. While both groups experienced equivalent HbA1c reductions relative to placebo, pramlintide-treated patients experienced reductions in postprandial glucose excursions and weight, not achievable with insulin therapy alone (Diabetes Care 2006; 29:2189-2195).

GIP and GLP-1 are the dominant peptide incretins responsible for the majority of nutrient-stimulated insulin secretion. Table 2 is a list of non-limiting examples of GLP-1 analogs. The insulinotropic effect of GLP-1 is strictly glucose dependent. GLP-1 stimulates all steps of insulin biosynthesis as well as insulin gene transcription. GLP-1 has tropic effects on B-cells. It stimulates B-cell proliferation and enhances the differentiation of new B-cells from progenitor cells in the pancreatic duct epithelium. Patients with type H diabetes have significantly impaired GLP-1 secretion and impaired responsiveness of B-cells to GIP. GLP-1 fragments that have GLP-1 activity are also included herein as GLP-1.

The term “GLP-1 receptor agonist” includes every therapeutic drug that shows agonistic activity for the GLP-1 receptors as a mechanism of action. Specifically, the agonists include GLP-1 itself, GLP-1 analogs, and any synthetic peptides that show agonistic activity for the GLP-1 receptors. Exenatide (BYETTA®) is one of GLP-1 receptor agonists. Exenatide (BYETTA®) is a synthetic peptide with 39-amino acid and has GLP-1-mimetic actions. Exenatide enhances glucose-dependent insulin secretion by the pancreatic beta-cell, suppresses inappropriately elevated glucagon secretion, and slows gastric emptying. Exenatide differs in chemical structure and pharmacological action from insulin, sulfonylureas, biguanides, thiazolidinediones, and alpha-glucosidase inhibitors. Exenatide has following mechanism of action by acting as GLP-1-mimetic: (1) Glucose-dependent insulin secretion: Exenatide has acute effects on pancreatic beta-cell responsiveness to glucose and leads to insulin release only in the presence of elevated glucose concentrations. This insulin secretion subsides as blood glucose concentrations decrease and approach euglycemia; (2) Glucagon secretion: In patients with type 2 diabetes, Exenatide moderates glucagon secretion and lowers serum glucagon concentrations during periods of hyperglycemia. Lower glucagon concentrations lead to decreased hepatic glucose output and decreased insulin demand. However, Exenatide does not impair the normal glucagon response to hypoglycemia; (3) Gastric emptying: Exenafide slows gastric emptying, thereby reducing the rate at which meal-derived glucose appears in the circulation; (4) Food intake: In both animals and humans, administration of Exenatide has been shown to reduce food intake. Many other GLP-1 receptor agonists are under development, including, but not limited to, liraglutide (NN-2211, NN2211, NNC-90-1170), betatropin (AC-2592), CJC-1131, insulinotropin, ITM-077 (BIM-51077, R-1583), ZP-10A (ZP-10, AVE-0010), PC-DAC: Exendin-4 (CJC-1134-PC).

Leptin is a 16 kD a protein hormone that plays a key role in regulating energy intake and energy expenditure, including the regulation of appetite and metabolism. The effects of leptin were observed by studying mutant obese mice that arose at random within a mouse colony at the Jackson Laboratory in 1950. These mice were massively obese and hyperphagic. Leptin itself was discovered in 1994 by Jeffrey M Friedman and colleagues at the Rockefeller University through the study of these mutant mice. The Ob(Lep) gene (Ob for obese and Lep for leptin) is located on chromosome 7 in humans. Leptin is produced by adipose tissue and interacts with six types of receptors (LepRa-LepRf). LepRb is the only receptor isoform that contains active intracellular signaling domains. This receptor is present in a number of hypothalamic nuclei, where it exerts its effects. Importantly, leptin binds to the Ventral Medial nucleus of the hypothalamus, known as the “satiety center.” Binding of leptin to this nucleus signals to the brain that the body has had enough to eat that is to say a sensation of satiety. A very small number of humans possess a mutant leptin gene. These people eat nearly constantly and may be more than 45 kg (100 pounds) overweight by the age of 7. Thus, circulating leptin levels give the brain a reading of energy storage for the purposes of regulating appetite and metabolism. Leptin works by inhibiting the activity of neurons that contain neuropeptide Y (NPY) and agouti-selated peptide (AgRP) and by increasing the activity of neurons expressing α-melanocyte-stimulating hormone (α-MSH). The NPY neurons are a key element in the regulation of appetite. Small doses of NPY injected into the brains of experimental animals stimulate feeding, while selective destruction of the NPY neurons in imice causes them to become anorexic. Conversely, α-MSH is an important mediator of satiety, and differences in the gene for the receptor at which α-MSH acts in the brain are linked to obesity in humans.

Adiponectin was first characterized in mice as a transcript over expressed in preadipocytes (precursors of fat cells) that differentiates into adipocytes. The human homologue was identified as the most abundant transcript in adipose tissue. Contrary to expectations, despite being produced in adipose tissue, adiponectin was found to be decreased in obesity. This down regulation has not been fully explained. The gene was localized to chromosome 3p27, a region highlighted as affecting genetic susceptibility to type 2 diabetes and obesity. Supplementation by different forms of adiponectin was able to improve insulin control, blood glucose and triglyceride levels in mice models. The gene was investigated for variants that predispose to type 2 diabetes. Several single nucleotide polymorphisms in the coding region and surrounding sequence were identified from several different populations, with varying prevalence, degrees of association and strength of effect on type 2 diabetes.

Insulin resistance is the condition in which normal amounts of insulin are inadequate to produce a normal insulin response from fat, muscle and liver cells. Insulin resistance in fat cells results in hydrolysis of stored triglycerides, which elevates free fatty acids in the blood plasma. Insulin resistance in muscle reduces glucose uptake whereas insulin resistance in liver reduces glucose storage, with both effects serving to elevate blood glucose. High plasma levels of insulin and glucose due to insulin resistance often leads to metabolic syndrome and type 2 diabetes.

Amounts of drugs administered to patients according to the present invention should be amounts effective to control blood sugar levels and diabetes mellitus to suitable levels. These amounts will vary according to the subject patient and can be determined by those of ordinary skill in the art. These amounts will vary by stage of disease, age, sex, weight, and the like of the patient. A positive effect of a drug is an effect that is desirable in controlling blood sugar and diabetes mellitus or an effect that is better than or improved over a previous effect in the same patient. A negative effect of a drug is an effect that is undesirable in controlling blood sugar and diabetes mellitus or an effect that is worse than or equal to a previous effect in the same patient.

The term “alpha-glucosidase inhibitor (AGI)” includes every therapeutic drug that shows inhibitory activity for membrane-bound intestinal alpha-glucoside hydrolase enzymes. Table 3 lists non-limiting examples of alpha-glucosidase inhibitors. For example, AGIs include, but not limiting to, voglibose (Basen), miglitol (Seiblue), acarbose (Glucobay), emiglitate, MDL-25637 and Luteolin. AGIs are useful drugs for oral treatment of postprandial hyperglycemia in patients suffering from type 2 diabetes mellitus. Inhibition of the enzyme in the brush border of the small intestine results in a delayed glucose absorption and a lowering of postprandial hyperglycemia.

The term “dipeptidyl peptidase IV (DPP-IV) inhibitor” includes every therapeutic drug that shows inhibitory activity for DPP-IV. Table 4 lists non-limiting examples of dipeptidyl peptidase IV inhibitors. DPP-IV inhibitors include, but are not limited to, sitagliptin (Januvia), vildagliptin (Galvas), alogliptin benzoate (SYR-322), saxagliptin (BMS-477118), denagliptin (Redana), Ondero (BI-1356), denagliptin (GW-823093C), DPP-728, P32/98, PSN-9301, MP-513, TA-6666, PHX-1149T, melogliptin (GRC-8200), R-1579, KRP-104, TS-021, GW-825964, 815541 and SSR-162369. DPP-IV inhibitor is believed to exert its actions in patients with type 2 diabetes by slowing the inactivation of incretins. When concentrations of the active intact incretins are increased by DPP-IV inhibitors, the actions of these hormones including GLP-1 and glucose-dependent insulinotropic polypeptide (GIP) are increased and prolonged. Functions of GLP-1 relating to the treatment of diabetic patients have been described on a previous page.

The term “insulin secretagogue” includes every therapeutic drug that has a mechanism of stimulating release of insulin from the pancreas as mechanism of action. Table 5 lists non-limiting examples of insulin secretagogues. The typical drugs are classified in glinides because they have a common molecular structure in the compounds. But, glinides are chemically unrelated to the oral sulfonylurea insulin secretagogues. Glinides are an oral blood glucose-lowering drug used in the management of type 2 diabetes mellitus and include, but not limiting to, repaglinide (Prandin, NovoNorm, GlucoNorm, Actulin), nateglinide (Starsis, Fastic, Starlix, Trazec) and mitiglinide (Glinsuna, Glufast). Mechanism of action for repaglinide is as follows: Repaglinide lowers blood glucose levels by stimulating the release of insulin from the pancreas. This action is dependent upon functioning beta (β) cells in the pancreatic islets. Insulin release is glucose-dependent and diminishes at low glucose concentrations. Repaglinide closes ATP-dependent potassium channels in the B-cell membrane by binding at characterized sites. This potassium channel blockade depolarizes the β-cell, which leads to an opening of calcium channels. The resulting increased calcium influx induces insulin secretion. The ion channel mechanism is highly tissue selective with low affinity for heart and skeletal muscle. Many other insulin secretagogues are under development, including, but are not limited to, Adyvia, JTT-608, Asterin, Myrtillin and Lupanin.

### EXAMPLES

The following examples are non-limiting.

### Example 1

1,5-AG was assessed as a marker of post-prandial blood glucose (PPG) control in pramlintide-treated patients with type I diabetes (TIDM). PPG is the glucose that appears in the blood stream and tissues after a meal. PPG predominates in the serum over average fasting glucose at HbA1c's less than 8.5%. Antihyperglycemic drugs affect PPG.

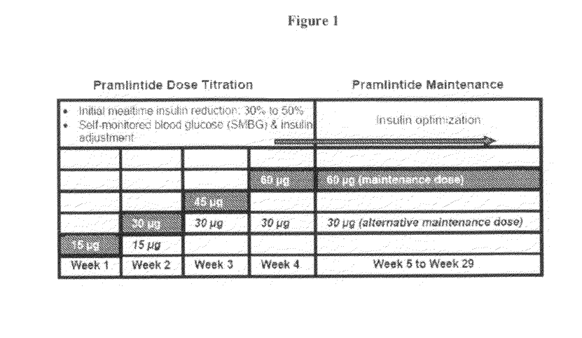

Post-hoc analysis of a randomized, double-blind, placebo-controlled study of a subset of subjects with T1DM on intensive insulin therapy with a baseline HbA1c≦8% (N=37, age 40±12 y; HbA1c 7.5±0.3%; weight 85.9±20.8 kg; mean±SD) treated with pramlintide (30/60 μg) or placebo with major meals. The study design is shown in FIG. 1.

### Endpoints

HbA1c, weight, and insulin dose measured at scheduled visits
Pre-prandial and post-prandial self-monitored blood glucose (SMBG) daily
Plasma 1,5-AG (GlycoMark assay) measured at baseline and week 29

### Statistical Analysis

All evaluable subjects with a baseline HbA1c≦8% and 1,5-AG measured at baseline and week 29
Mean (±SE) change from baseline HbA1c, body weight, PPG, insulin use and 1,5-AG at week 29
A repeated measures analysis across all study visits was performed comparing pramlintide and placebo groups

Table 6 compares the baseline characteristics of patients treated with either a placebo or pramlintide.

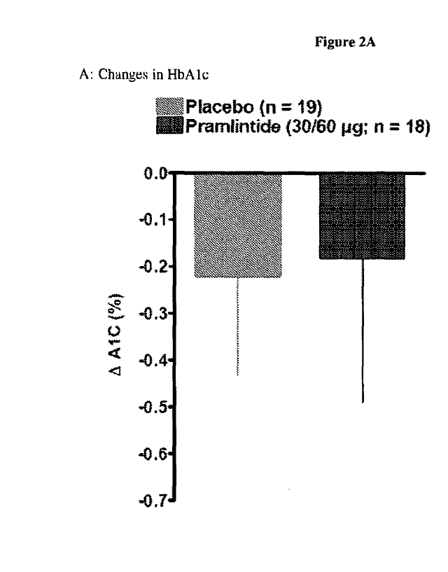

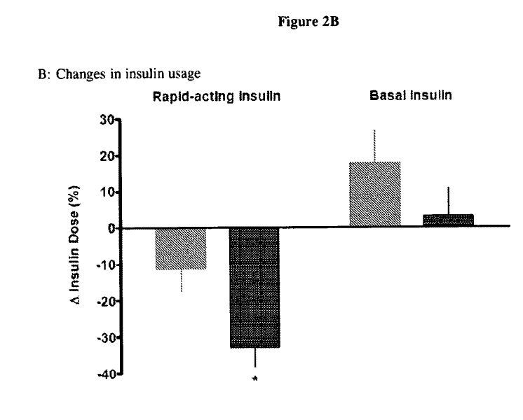

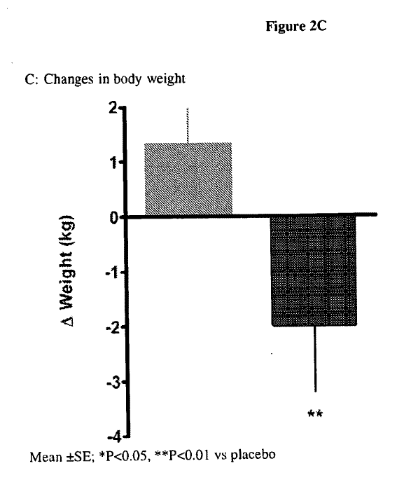

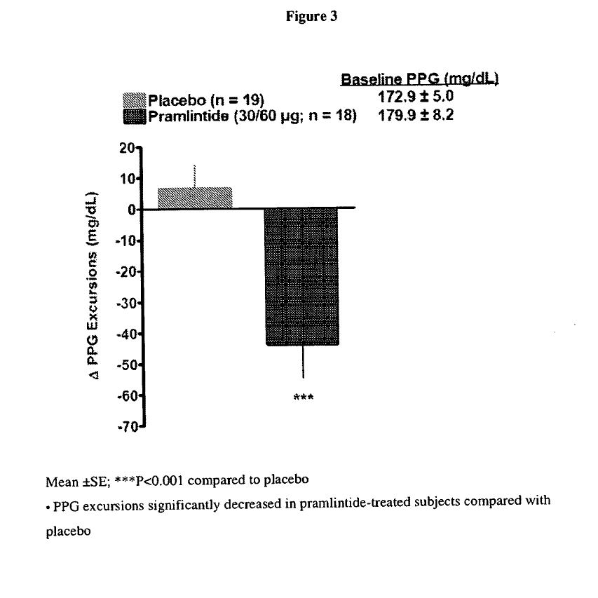

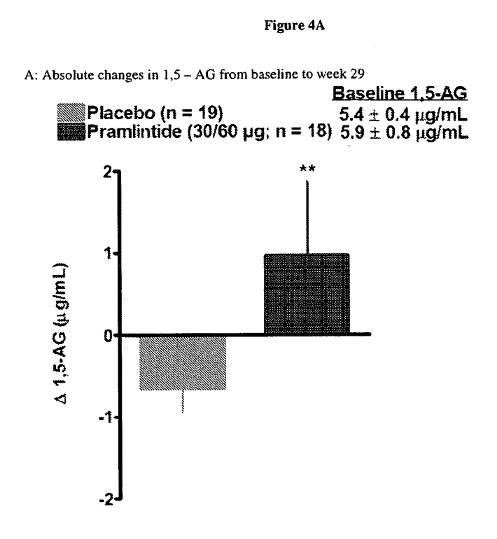

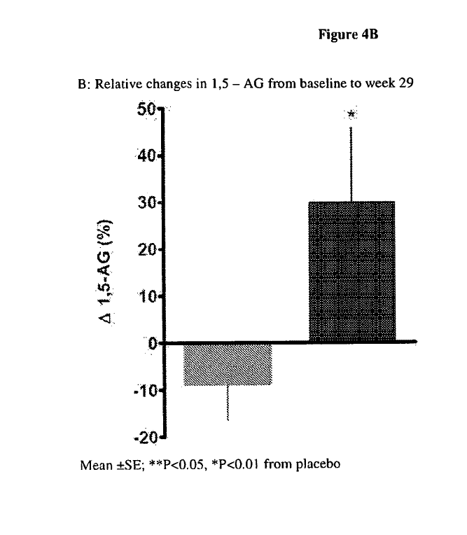

A repeated measures analysis across all visits was performed comparing pramlintide and placebo groups. Subjects in both groups targeted similar glycemic goals. The results of this study are presented in FIGS. 2, 3 and 4. Table FIG. 2 A, B, and C—show the changes in HbA1c, insulin use and body weight from baseline to week 29. FIG. 3—show changes in PPG excursions from baseline at week 29. FIGS. 4 A and B demonstrate the absolute and relative changes in 1,5-AG from baseline to week 29. Table 7 summarizes the parameter changes in patients with HbA1c less than or equal 8.0% (P-values are by T test.)

At week 29, pramlintide (n=18) improved 2 hr PPG excursions* (−43.9±10.9 vs +6.5±7.6 mg/dL, P<0.001; mean±SE), reduced body weight (−2.0±1.2 vs +1.3±0.7 kg, P<0.01), and resulted in similar reductions in HbA1c (−0.18±0.31 vs. −0.22±0.21%) compared with placebo (n=19). Consistent with the improvement in PPG, fasting plasma 1,5-AG levels increased significantly from baseline to wk 29, relative to placebo (+0.96±0.91 vs −0.65±0.41 μg/mL, P<0.05; +30±16% vs −9±8%, P<0.01). The most common adverse event associated with pramlintide use was mild to moderate nausea. *“2 hour excursions”. This refers simply to blood glucose levels two hours after a meal. This is the increase in glucose at two hours that results from consumption of various sources of glucose.
At week 29, pramlintide- and placebo-treatment resulted in similar reductions in HbA1c, while mealtime insulin use significantly decreased in pramlintide-treated subjects
Body weight significantly decreased in pramlintide-treated subjects after 29 weeks of treatment compared to an increase in body weight in placebo-treated subjects
PPG excursions significantly decreased in pramlintide-treated subjects compared with placebo
At week 29, 1,5-AG levels increased significantly in pramlintide-compared to placebo-treated subjects
In this post-hoc analysis in moderately well-controlled subjects with type 1 diabetes, pramlintide, as an adjunct treatment for subjects on intensive insulin therapy led to:
Improved postprandial glucose control
Significantly reduced body weight
Despite similar reductions in HbA1c, the change in 1,5-AG levels was consistent with the improvement in PPG control in pramlintide-treated subjects, as measured by SMBG
1,5-AG, as a complement to HbA1c, may be a useful marker of PPG control

These results are consistent with the biology of the GlycoMark™ 1,5-AG assay which reflects glucose levels above the renal threshold of glucosuria, As postprandial glucose levels predominate in the lower HbA1c ranges, the 1,5-AG assay reflects elevated post-meal glucose levels more accurately. The 1,5-AG assay is reflective of differing post-meal glucose levels, despite similarities in HbA1c values in moderately controlled patients (HbA1c<8.0).

It should also be noted in this analysis that the primary differentiating variable between the treatment groups is glucose excursion change. The 1,5-AG assay correlates significantly to glucose excursions (r=0.21, p<0.01) and correlates more significantly to postmeal glucose levels as HbA1c levels decrease (in fact, when partial correlations are calculated between the 1,5-AG assay post-meal glucose levels in which HbA1c values are held constant, the r value is 0.20, p<0.01). The correlation of excursions to the 1,5-AG assay (no correlation of excursions to HbA1c), may explain why the 1,5-AG assay is able to differentiate the pramlintide and placebo groups. Thus, 1,5-AG levels may be reflective of glycemic variability and pramlintide's primary effect is on the reduction of glycemic variability.

### Conclusions:

Pramlintide, as an adjunct treatment for T1DM patients on intensive insulin therapy, led to improved PPG and significant reduction in body weight.
Despite similar reductions in HbAc, the change in 1,5-AG levels was consistent with improvement in PPG control in pramlintide-treated subjects, as measured by SMBG.
1,5-AG, as a complement to A1C, may be a useful marker of PPG control.

### Example 2

In this post-hoc analysis of a randomly selected subset of patients with type 2 diabetes mellitus (T2DM) with evaluable samples from three placebo-controlled studies (N=144; age 57.2±10.0 y; HbA1c 8.2±1.0%; weight 96.4±20.9 kg; mean±SD), plasma 1,5-AG was measured in patients treated for 30 weeks with either Exenatide (5 or 10 μg) or placebo.

The study design is depicted in FIG. 5.

The demographics and baseline characteristics of the study group are presented in Table 8.

Inclusion criteria for the placebo-controlled trials were:
Subjects with type 2 diabetes age 16 to 75 years
Treated for 3 months prior to screening with ≧1500 mg/day metformin and/or maximally-effective sulfonylurea dose
HbA1c 7.1% to 11.0%
FPG<240 mg/dL
BMI 27 to 45 kg/m²
Stable body weight (±10%) for 3 months prior to screening
No clinically relevant abnormal laboratory test values
No treatment with other anti-diabetes agents or weight loss drugs within prior 3 months.

Descriptive statistics for all subjects are provided for demographics, safety variables by treatment and pharmacodynamic parameters (1,5-AG, HbA1c, FPG, body weight) by treatment. Pearson correlation analysis is used between change in 1,5-AG value and change in HbA1c or FPG.

The results of this study to assess the utility of 1,5-anhydro-D-glucitol, HbA1c and fructosamine to demonstrate the efficacy of exenatide is presented in Table 9. Changes in 1,5 AG were significantly correlated with HbA1c change from baseline and FPG change from baseline. At both 5 μg and 10 μg dosages only 1,5-AG moved significantly, compared to the placebo group of patients, after a six month course of therapy with Exenatide at both 5 μg and 10 μg dosages. 1,5-AG changed 2.7+/−0.6 μg/ml (p<0.05) and 2.9+/−0.6 μg/ml (p<0.01) from baseline with 5 μg of and 10 μg of Exenatide, respectively. HbA1c showed a significant (p<0.01) change from baseline −0.9+/−0.1% with 10 μg of Exenatide but no significant change with 5 μg of the drug. Fructosamine showed non significant movement with either dosage.

### Conclusions:

Previous studies have shown that as HbA1c nears 7%, PPG becomes the major contributor to overall glycemic control. As such, 1,5-AG may be a useful complement to HbA1C to reflect PPG in patients with T2DM treated with agents that target PPG. In this post-hoc analysis, the increase in 1,5-AG confirms previously reported improvements in PPG in Exenatide-treated patients (Bhole, D. et al. Exenatide Improves Postprandial Glucose Control in Patients with Type 2 Diabetes, as Measured by 1,5-Anhydroglucitol (GlycoMark). Exenatide GlycoMark Abstract EASD, 2007).

All patents, patent applications, literature, and test methods mentioned herein are hereby incorporated-by-reference as if fully repeated herein. Other variations of the present invention may be discerned form the above detailed description. All such obvious variations are within the scope of the present invention.

Table 1 lists non-limiting examples of amylin analogs.

| Drug Name | Chemical Name/Description | Company |
|---|---|---|
| Pramlintide | L-Lysyl-L-cysteinyl-L-asparaginyl-L-threonyl-L-alanyl-L-threonyl-L-cysteinyl-L-alanyl-L-threonyl-L-glutaminyl-L- | Amylin |
| acetate, Normylin, | arginyl-L-leucyl-L-alanyl-L-asparaginyl-L-phenylalanyl-L-leucyl-L-valyl-L-histidyl-L-seryl-L-seryl-L-asparaginyl-L- | |
| Symlin. AC-137, | phenylalanyl-glycyl-L-protyl-L-isoleucyl-L-leucyl-L-protyl-L-threonyl-L-asparaginyl-L-valyl-L-glycyl-L-seryl-L- | |
| AC-0137, | prollneamylin (human) acetate hydrate | |
| Triproamylin | | |
| acetate | | |
| [35Glu,40Glu] | [35Glu,40Glu]Amylin family polypepttde-6 (1-47); L-Threonyl-L-glutaminyl-L-alanyl-L-glutaminyl-L-leucyl-L-leucyl-L- | Amylin |
| AFP-6(1-47) | arginyl-L-valyl-L-glycyl-L-cysteinyl-L-valyl-L-leucyl-glycyl-L-threonyl-L-cysteinyl-L-glutaminyl-L-asparaginyl-L- | |
| | leucyl-L-seryl-L-histidyl-L-arginyl-L-leucyl-L-tryptophyl-L-glutaminyl-L-leucyl-L-methionyl-glycyl-L-prolyl-L-alanyl- | |
| | glycyl-L-arginyl-L-glutaminyl-L-glutamyl-L-seryl-L-alanyl-L-prolyl-L-valyl-L-glutamyl-L-prolyl-L-seryl-L-seryl-L- | |
| | prolyl-L-histidyl-L-tryosinamide | |
| [25Wdel]AFP- | L-Valyl-L-glutaminyl-L-asparaginyl-L-leucyl-L-seryl-L-histidyl-L-arginyl-L-leucyl-L-glutaminyl-L-leucyl-L-methinyl- | Amylin |
| 6(17-47) | glycyl-L-prolyl-L-alanyl-glycyl-L-arginyl-L-glutaminyl-L-aspartyl-L-seryl-L-alanyl-L-prolyl-L-valyl-L-aspartyl-L- | |
| | prolyl-L-seryl-L-seryl-L-prolyl-L-histidyl-L-seryl-L-tyrosinamide; [25Wdel]Amylin family polypeptide-6 (17-47) | |
| | H-Lys-Cys-Asn-Thr-Ala-Thr-Cys-Ala-Thr-Gln-Arg-Leu-Ala-Phe-Leu-Val-Arg-Ser-Ser-Asn-Phe-Gly-Pro-Ile-Ile-Leu-Pro-Ser-Thr- | Amylin |
| | Asn-Val-Gly-Ser-Asn-Thr-Tyr-NH2 cyclic disulfide | |
| | H-Cys-Asn-Thr-Ala-Thr-Cys-Ala-Thr-Gln-Arg-Leu-Ala-Phe-Leu-Val-Arg-Ser-Ser-Asn-Asn-Phe-Gly-Pro-Ile-Leu-Pro-Ser-Thr-Asn- | Amylin |
| | Val-Gly-Ser-Asn-Thr-Tyr-NH2 cyclic disutfide | |
| | H-Lys-Cys-Asn-Thr-Ala-Thr-Cys-Ala-Thr-Gln-Arg-Leu-Ala-Asn-Phe-Leu-Val-Arg-Ser-Ser-Asn-Phe-Gly-Pro-Ile-Leu-Pro-Pro-Thr- | Amylin |
| | Asn-Val-Gly-Ser-Asn-Thr-Tyr-NH2 cyclic disulfide | |
| | H-Cys-Asn-Thr-Ala-Thr-Cys-Ala-Thr-Gln-Arg-Leu-Ala-Phe-Leu-Val-Arg-Ser-Ser-Asn-Asn-Phe-Gly-Pro-Pro-Thr-Asn-Val-Gly-Ser- | Amylin |
| | Asn-Thr-Tyr-NH2 cyclic disulfide | |
| | H-Cys-Asn-Thr-Ala-Thr-Cys-Ala-Thr-Gln-Arg-Leu-Ala-Asn-Phe-Val-His-Ser-Ser-Asn-Phe-Gly-Pro-Ile-Leu-Pro-Pro-Thr-Asn-Val- | Amylin |
| | Gly-Ser-Asn-Thr-Tyr-NH2 cyclic disulfide | |
| | H-Lys-Cys-Asn-Thr-Ala-Thr-Cys-Ala-Thr-Gln-Arg-Leu-Ala-Asn-Phe-Leu-Val-His-Ser-Ser-Asn-Asn-Phe-Gly-Ala-Ile-Leu-Pro-Ser- | Amylin |
| | Thr-Asn-Val-Gly-Ser-Asn-Thr-Tyr-NH2 cyclic disulfide | |
| | L-Lysyl-L-cysteinyl-L-asparaginyl-L-threonyl-L-alanyl-L-threonyl-L-cysteinyl-L-alanyl-L-threonyl-L-alanyl-L-arginyl-L- | Amylin |
| | leucyl-L-alanyl-L-alanyl-L-phenylalanyl-L-leucyl-L-alanyl-L-arginyl-L-seryl-L-seryl-glycyl-L-tyrosinamide cyclic | |
| | (S-3.2-S-3.7)-disulfide | |
| | L-Cysteinyl-L-asparaginyl-L-threonyl-L-alanyl-L-threonyl-L-cysteinyl-L-alanyl-L-threonyl-L-alanyl-L-arginyl-L-leucyl- | Amylin |
| | L-alanyl-L-alanyl-L-phenylalanyl-L-leucyl-L-alanyl-L-arginyl-L-serinamide cycllc (S-3.1-S-3.6)-disulfide | |
| | L-Lysyl-L-cysteinyl-L-asparaginyl-L-threonyl-L-alanyl-L-threonyl-L-cysteinyl-L-alanyl-L-threonyl-L-alanyl-L-arginyl- | Amylin |
| | L-leucyl-L-alanyl-L-asparaginyl-L-phenylalanyl-L-leucyl-L-valyl-L-arginyl-L-seryl-L-seryl-L-glycyl-L-tyrosinamide | |
| | cyclic (S-3.2-S-3.7)-disulfide | |
| | L-Leucyl-L-seryl-L-threonyl-L-alanyl-L-alanyl-L-threonyl-L-cysteinyl-L-alanyl-Lthreonyl-L-alanyl-L-arginyl-L-leucyl- | Amylin |
| | L-alanyl-L-arginyl-L-serinamide | |
| | L-Cysteinyl-L-asparaginyl-L-threonyl-L-alanyl-L-threonyl-L-cysteinyl-L-alanyl-L-threonyl-L-glutaminyl-L-arginyl-L- | Amylin |
| | leucyl-L-alanyl-Lasparaginyl-L-phenylalanyl-L-leucyl-L-valyl-L-arginyl-L-seryl-L-seryl-L-glycyl-L-tyrosinamide cydic | (Originator); |
| | (S-3.1-S-3.6)-disulfide | |
| | L-Lysyl-L-cysteinyl-L-asparaginyl-L-threonyl-L-alanyl-L-threonyl-L-cysteinyl-L-alanyl-L-threonyl-L-alanyl-L-arginyl-L- | Amylin |
| | leucyl-L-alanyl-L-asparaginyl-L-phenylalanyl-L-leucyl-L-valyl-L-arginyl-L-seryl-L-seryl-glycyl-L-tyrosinamide cyclic | (Originator); |
| | (S-3.2-S-3.7)-disulfide | |
| | L-Lysyl-L-cysteinyl-L-asparaginyl-L-threonyl-L-alanyl-L-threonyl-L-cysteinyl-L-alanyl-L-threonyl-L-glutaminyl-L- | Novo Nordisk |
| | arginyl-L-leucyl-L-alanyl-L-asparaginyl-L-phenylalanyl-L-leucyl-L-valyl-N6-[17-(N-hexadecanoyl-DL-gamma- | |
| | glutamylamino)-10-oxo-3,6,12,15-tetraoxa-9-azaheptadecanoyl]-L-lysyl-L-seryl-L-seryl-l-asperaginly-L-asparaginyl-L- | |
| | leucyl-glycyl-L-prolyl-L-valyl-L-leucyl-L-prolyl-L-prolyl-L-threonyl-L-asparaginyl-L-valyl-glycyl-L-seryl-L- | |
| | asparaginyl-L-threonyl-L-tyrosinamide cyclic disulfide | |

Table 2 lists non-limiting examples of GLP-1 analogs.

| Drug Name | Company |
|---|---|
| Exenatide, Exenatide LAR, Exendin-4, Byetta, AC-2993, LY-2148568, | Lilly, Nastech, Amylin, Alkermes |
| AC-2993 LAR | |
| Liraglutide, NNC-90-1170, NN-2211, NN2211 | Novo Nordisk |
| GLP-1, Glucagon-like peptide 1, Insulinotropin | Roche, Scios, Novo Nordisk |
| DAC:GLP-1, CJC-1131 | ConjuChem |
| ZP-10A, AVE-0010, ZP-10 | Zealand Pharmaceuticals, sanofi-aventis |
| ITM-077, BIM-51071, R-1583 | Teijin Pharma, Chugai Pharmaceutical, Roche, Ipsen, |
| | SCRAS |
| Betatropin, AC-2592, GLP-1(7-36)amide | Amylin |
| Albiglutide, Syncria, Albugon, PGC GLP-1, GSK-716155 | GlaxoSmithKline, Human Genome Sciences |
| CJC-1134-PC, PC-DAC, Exendin-4 | ConjuChem |
| TT-223/GLP1, GLP1-INT | Transition Therapeutics |
| CS-872, SUN-E7001, rGLP-1(7-36)amide | Daiichi Sankyo, Asubio |
| TH-0318, ThGLP-1 | Theratechnologies, OctoPlus |
| L-Histidyl-L-alanyl-L-glutamyl-glycyl-L-threonyl-L-phenylalanyl-L- | Novo Nordisk |
| threonyl-L-seryl-L-aspartyl-L-valyl-L-seryl-L-seryl-L-tyrosyl-L-leucyl-L- | |
| glutamyl-glycyl-L-glutaminyl-L-alanyl-L-alanyl-L-arginyl-L-glutamyl-L- | |
| phenylalanyl-L-isoleucyl-L-alanyl-L-tryptophyl-L-leucyl-L-valyl- | |
| Nepsilon-(Nalpha-hexadecanoyl-gamma-L-glutamyl)-L-lysyl-glycyl-L- | |
| arginyl-glycine | |
| [Aib(8,35)]hGLP-1(1-36)NH2 | Biomeasure |
| PEG-DAPD | Bayer |
| CNTO-736 | Centocor |
| CVX-73, CVX-073 | CovX |
| CVX-98, CVX-098 | CovX |
| CVX-096 | CovX |
| PGC-GLP-1, PGC-HC/GLP-1, PGC-HC formulated GLP-1, PGC-HC- | PharmalN |
| E/GLP-1 | |
| Exendin-4(PEAPTD)2 mTF | BioRexis, Pfizer |

Table 3 lists non-limiting examples of alpha-glucosidase inhibitors.

| Drug Name | company |
|---|---|
| Bay-g-5421, Acarbose, Precose, | Bayer |
| Glucobay, Glucor, Prandase | |
| A-71100, AO-128, Voglibose, | Takeda |
| Basen OD, Glustat, Basen | |
| SK-983, Bay-m-1099, Miglitol, Glyset, | Bayer, Sanwa, Pfizer, Lacer, |
| Seibule, Diastabol, Plumarol | sanofi-aventis |
| Bay-o-1248, MKC-542, Emiglitate | Bayer, Mitsubishi Tanabe |
| | Pharma |
| MDL-25637 | sanofi-aventis |
| Luteolin | Institute of Materia Medica, |
| | Beijing, Chinese Academy |
| | of Medical Sciences |

Table 4 lists non-limiting examples of dipeptidyl peptidase IV inhibitors.

| Drug Name | Company |
|---|---|
| ONO-5435, MK-431, MK-0431, | Merck & Co., Merck Sharp & |
| Sitagliptin phosphate monohydrate, | Dohme, Banyu, Ono |
| Januvia, Tesavel, Glactiv, Xelevia | |
| LAF-237, NVP-LAF-237, | Novartis |
| Vildagliptin, Galvus | |
| SYR-322, Alogliptin benzoate | Takeda |
| BMS-477118, BMS-477118-11 | AstraZeneca, Bristol-Myers |
| (monohydrate), Saxagliptin | Squibb, Otsuka |
| (monohydrate) | |
| GW-823093, 823093, Denagliptin, | GlaxoSmithKline |
| Redona | |
| BI-1356, BI-1356-BS, Ondero | Boehringer Ingelheim |
| GW-823093C, Denagliptin tosilate | GlaxoSmithKline |
| DPP-728, NVP-728, NVP-DPP-728 | Novartis |
| P32/98 | Probiodrug, Merck & Co. |
| P93/01, P-93/01, PSN-9301 | OSI Prosidion, Probiodrug, OSI |
| MP-513 | Mitsubishi Tanabe Pharma |
| T-6666, TA-6666 | Mitsubishi Tanabe Pharma |
| PHX-1149, PHX-1149T | Phenomix Corp. |
| EMD-675992, GRC-8200, Melogliptin | Glenmark Pharmaceuticals |
| R-1579 | Roche |
| KRP-104 | ActivX; Kyorin |
| TS-021 | Taisho |
| 825964, GW-825964 | GlaxoSmithKline |
| 815541 | GlaxoSmithKline |
| SSR-162369 | sanofi-aventis |

Table 5 lists non-limiting examples of insulin secretagogues.

| Drug Name | Company |
|---|---|
| SMP-508, AG-EE-623 ZW, NN-623, AG-EE-388 (racemate), | Fournier, Daiichi Sankyo, Sciele, Takeda, Boehringer Ingelheim, |
| Repaglinide (racemate), Prandin, NovoNorm, GlucoNorm, | Menarini, Dainippon Sumitomo Pharma, Novo Nordisk |
| Actulin | |
| DJN-608, A-4166, AY-4166, SDZ-DJN-608, YM-026, | Ajinomoto (Originator), Astellas Pharma, Daiichi Sankyo, II-Dong, |
| Nateglinide, Starsis, Fastic, Starlix, Trazec | Novartis |
| KAD-1229, S-21403, Mitiglinide calcium hydrate, Glinsuna, | Kissei, USV, Servier, Choongwae, Takeda, Eisai, Elixir |
| Glufast | Pharmaceuticals |
| NN-4440, Repaglinide/metformin hydrochloride, PrandiMet | Sciele, Novo Nordisk |
| JD-1101, 4-OH-Ile, Adyvia | Innodia |
| JTT-608 | Japan Tobacco |
| Cyanidin-3-glucoside, Chrysanthemin, Cyanidin 3-O-beta-D- | Sigma-Aldrich, Kyung Hee University, Michigan State University |
| glucopyranoside, Chrysontemin, Asterin, Glucocyanidin, | |
| Kuromanine | |
| Delphinidin-3-glucoside, Myrtillin | Sigma-Aldrich, Kyung Hee University, Michigan State University |
| Lupanine, (+)-2-Oxosparteine, Lupanin | |

TABLE 6
Baseline characteristics

| Evaluable N = 37 | Placebo (n = 19) | Pramlintide (n = 18) |
|---|---|---|
| Age (y) | 41 ± 11 | 40 ± 13 |
| Duration of diabetes (y) | 17 ± 10 | 18 ± 8 |
| HbA1c (%) | 7.5 ± 0.3 | 7.6 ± 0.4 |
| Weight (kg) | 87.4 ± 19.2 | 84.4 ± 22.9 |
| BMI (kg/m2) | 28.9 ± 5.5 | 27.9 ± 5.8 |
| Total daily mealtime | 30.9 ± 15.0 | 31.8 ± 22.8 |
| insulin (units) | | |
| Total daily basal insulin | 27.8 ± 12.7 | 37.6 ± 26.8 |
| (units) | | |
| Total daily insulin (units) | 58.7 ± 24.1 | 69.4 ± 45.4 |

Data are Mean ± SD

TABLE 7
Change of parameters in patient with HbA1c less than or equal 8.0% (P-values are by T test.)

| | | Plasma 1,5-AG (cters | | | HbA1c (%) | | | | | | | | |
|---|---|---|---|---|---|---|---|---|---|---|---|---|---|
| | | | | p- | | | p- | PPG (mg/dL) | | | Body Weight (kg) | | |
| | | Placebo | Pramlintide | value | Placebo | Pramlintide | value | Placebo | Pramlintide | p-value | Placebo | Pramlintide | p-value |
| Baseline | Mean | 5.4 | 5.9 | — | 7.5 | 7.6 | — | 173 | 180 | — | 86.5 | 85.0 | — |
| | | (n = 19) | (n = 18) | | (n = 19) | (n = 18) | | | | | | | |
| 4 weeks | Mean | 5.4 | 6.4 | — | 7.1 | 7.0 | — | 181 | 143 | — | 86.1 | 84.2 | — |
| | | (n = 19) | (n = 18) | | (n = 19) | (n = 18) | | | | | | | |
| | % | 0.4 | 15.9 | 0.13 | −6.3 | −7.4 | 0.30 | 5.1 | −18.9 | <0.01 | −0.4 | −0.7 | 0.62 |
| | Change | | | | | | | | | | | | |
| | from | | | | | | | | | | | | |
| | baseline | | | | | | | | | | | | |
| 16 weeks | Mean | 5.2 | 7.0 | — | 7.3 | 7.2 | — | 164 | 145 | — | 87.2 | 82.5 | — |
| | | (n = 17) | (n = 17) | | (n = 19) | (n = 16) | | | | | | | |
| | % | −0.3 | 23.4 | 0.12 | −6.9 | −7.0 | 0.69 | −4.1 | −16.8 | 0.11 | 0.8 | −2.8 | <0.01 |
| | Change | | | | | | | | | | | | |
| | from | | | | | | | | | | | | |
| | baseline | | | | | | | | | | | | |
| 29 weeks | Mean | 4.7 | 6.9 | — | 7.3 | 7.4 | — | 162 | 135 | — | 87.8 | 83.0 | — |
| | | (n = 19) | (n = 18) | | (n = 19) | (n = 18) | | | | | | | |
| | % | −8.8 | 29.8 | 0.03 | −6.1 | −4.5 | 0.88 | −6.2 | −27.2 | 0.04 | 1.4 | −2.2 | 0.04 |
| | Change | | | | | | | | | | | | |
| | from | | | | | | | | | | | | |
| | baseline | | | | | | | | | | | | |

TABLE 8
Demographic and baseline characteristics by treatment (N = 144)

| | | Exenatide | Exenatide | All |
|---|---|---|---|---|
| | Placebo | 5 μg | 10 μg | Subjects |
| | (n = 44) | (n = 42) | (n = 58) | (N = 144) |
| Sex, male/female (%) | 55/46 | 60/41 | 53/47 | 56/44 |
| Age (y) | 59 ± 9 | 57 ± 10 | 56 ± 1 | 57 ± 10 |
| Race, Caucasian/Black/ | 75/11/11/2 | 55/17/26/2 | 60/22/17/0 | 63/17/18/1 |
| Hispanic/Other (%)* | | | | |
| Body weight (kg) | 97 ± 21 | 96 ± 23 | 96 ± 19 | 96 ± 21 |
| BMI (kg/m²) | 33 ± 5 | 33 ± 7 | 34 ± 6 | 33 ± 6 |
| HbA1c (%) | 8.3 ± 1.1 | 7.9 ± 0.7 | 8.3 ± 1.1 | 8.2 ± 1.0 |
| FPG (mg/dL) | | | | |
| Duration of diabetes (y) | 7 ± 6 | 7 ± 7 | 7 ± 5 | 7 ± 6 |

Data are mean ± SD, except for sex and race;
*Due to rounding, percentages may not add up to 100.

TABLE 9
1,5-AG, HbA1c, FPG and body weight change from baseline (N = 144)

| | | Exenatide | Exenatide |
|---|---|---|---|
| | Placebo | 5 μg | 10 μg |
| | (n = 44) | (n = 42) | (n = 58) |
| 1,5-AG change from baseline | — | 2.7 ± 0.6* | 2.9 ± 0.6** |
| (μg/mL) | | | |
| 1,5-AG change from baseline | 26 ± 19 | 45.3 ± 11.9* | 69.4 ± 14.6** |
| (%) | | | |
| HbA1c change from baseline | −0.1 ± 0.1 | −0.5 ± 0.1 | −0.9 ± 0.1** |
| (%) | | | |
| FPG change from baseline | 10.7 ± 7.5 | −8.9 ± 7.5 | −4.4 ± 5.5 |
| (mg/dl) | | | |
| Body weight change from | −1.6 ± 0.6 | −2.3 ± 0.5 | −2.0 ± 0.4 |
| baseline (kg) | | | |

Mean ± SE;
*P < 0.05,
**P < 0.01 from baseline

1. A method for determining the effect of one or more antihyperglycemia diabetes treatment drugs on a person in need of such treatment, said method comprising:
(a) measuring the 1,5-anhydro-D-glucitol level of said patient to obtain a first 1,5-anhydro-D-glucitol level;
(b) administering said one or more antihyperglycemia drugs to said patient; and
(c) measuring the 1,5-anhydro-D-glucitol level of said patient after step (b) to obtain a second 1,5-anhydro-D-glucitol level;
wherein said effect of said one or more drugs is not reflected by mean hemoglobin A1c values; and
wherein an increase of said second 1,5-anhydro-D-glucitol level over said first 1,5-anhydro-D-glucitol level indicates a positive effect of said one or more drugs.

2. The method as defined in claim 1, wherein at least one of said one or more drugs is a peptide.

3. The method as defined in claim 1, wherein at least one of said one or more drugs is selected from the group consisting of amylin, an amylin receptor agonist, glucagon-like peptide 1 or an active fragment thereof, a glucogon-like peptide 1 receptor agonist, or any combination of any of the foregoing.

4. The method as defined in claim 1, wherein at least one of said one or more drugs is a non-peptide.

5. The method as defined in claim 1, wherein at least one of said one or more drugs is selected from the group consisting of alpha-glucosidase inhibitor, dipeptidyl peptidase IV inhibitor, or insulin secretagogue.

6. A method as defined in claim 1, wherein said patient is also undergoing insulin therapy.

7. A method of evaluating treatment by one or more drugs selected from the group consisting of amylin, an amylin receptor agonist, glucagon-like peptide 1 or an active fragment thereof, a glucogon-like peptide 1 receptor agonist of a patient suffering from diabetes mellitus, said method comprising:
(a) measuring the 1,5-anhydro-D-glucitol level of said patient to obtain a first 1,5-anhydro-D-glucitol level;
(b) administering said one or more drugs to said patient; and
(c) measuring the 1,5-anhydro-D-glucitol level of said patient after step (b) to obtain a second 1,5-anhydro-D-glucitol level;
wherein an increase of said second 1,5-anhydro-D-glucitol level over said first 1,5-anhydro-D-glucitol level indicates a positive effect of said one or more drugs.

8. The method as defined in claim 7, wherein said patient is also undergoing insulin therapy.

9. A method of regulating the desired dosage of one or more antihyperglycemia drugs selected from the group consisting of amylin, an amylin receptor agonist, glucagon-like peptide 1 or an active fragment thereof, a glucogon-like peptide 1 receptor agonist or any combination of any of the foregoing to be administered to a patient suffering from diabetes mellitus, said method comprising:
(a) administering a first predetermined dosage of said one or more drugs to said patient; and
(b) measuring the 1,5-anhydro-D-glucitol level of said patient after step (a) to obtain a first 1,5-anhydro-D-glucitol level;
(c) administering a second predetermined dosage of the same one or more drugs to said patient; and
(d) measuring the 1,5-anhydro-D-glucitol level of said patient after step (c) to obtain a first 1,5-anhydro-D-glucitol level:
wherein an increase of said second 1,5-anhydro-D-glucitol level over said first 1,5-anhydro-D-glucitol level indicates that said second predetermined dosage preferred over said first predetermined dosage for said patient.

10. The method as defined in claim 9, wherein said patient is also undergoing insulin therapy.
